(19) United States
(12) Patent Application Publication
(10) Pub. No.: US 2009/0018336 A1
(43) Pub. Date: Jan. 15, 2009

(54) Racemization process of R-zopiclone

(76) Inventors: Nina Finkelstein, Herzliya (IL); Marioara Mendelovici, Rehovot (IL)

Correspondence Address: KENYON & KENYON LLP, ONE BROADWAY, NEW YORK, NY, 10004, US

(21) Appl. No.: 12/215,249

(22) Filed: Jun. 25, 2008

Related U.S. Application Data
(60) Provisional application No. 60/929,375, filed on Jun. 25, 2007.
(60) Provisional application No. 61/056,157, filed on May 27, 2008.

(51) Int. Cl. C07D 471/04

(52) U.S. Cl. 544/350

(57) ABSTRACT

In one of the embodiments, the present invention provides a racemization process comprising: removing the organic solvent from a mother liquor comprising R-zopiclone malate, eszopiclone malate and an organic solvent to obtain a mixture of R-zopiclone malate and eszopiclone malate; mixing the mixture with water to obtain a solution; neutralizing R-zopiclone malate and eszopiclone malate in the solution to obtain a precipitate of R-zopiclone and eszopiclone; filtering the precipitate; mixing the precipitate of R-zopiclone and eszopiclone with DBU and an organic inert solvent having a boiling point of at least 80° C.; heating; and cooling to obtain a zopiclone racemate.

### CROSS REFERENCE TO RELATED APPLICATIONS

The present application claims benefits of U.S. Provisional Application No. 60/929,375 filed Jun. 25, 2007 and U.S. Provisional Application No. 61/056,157 filed May 27, 2008, the disclosures of both provisional applications are incorporated by reference.

### FIELD OF THE INVENTION

The present invention relates to a new method useful for preparing eszopiclone involving neutralization of a material comprising an acid addition salt of R-zopiclone or racemization of a material comprising R-zopiclone.

### BACKGROUND

Zopiclone is known as a non-benzodiazepine which can be used to induce a sedative, hypnotic or tranquilizing effect and therefore used for treating insomnia. Zopiclone is a racemate having a chemical name of 4-methyl-1-piperazinecarboxylic acid 6-(5-chloro-2-pyridinyl)-6,7-dihydro-7-oxo-5H-pyrrolo[3,4-b]pyrazin-5-yl ester, (±)-6-(5-chloro-2-pyridinyl)-6,7-dihydro-7-oxo-5H-pyrrolo[3,4-b]pyrazin-5-yl-4-methylpiperazine-1-carboxylate, or 6-(5-chloropyrid-2-yl)-5-(4-methylpiperazin-1-yl)carbonyloxy-7-oxo-6,7-dihydro-5H-pyrrolo[3,4-b]pyrazine. The molecule structure is described schematically in FIG. I.

Eszopiclone is the S-enantiomer of zopiclone, and is more active and less toxic than the racemic zopiclone according to U.S. Pat. No. 6,444,673 B1. This drug has been marketed in the United States by Sepracor™ under the name Lunesta®, formerly known as Estorra®, having CAS Registry Number is 138729-47-2. Eszopiclone has a chemical name of (+)-6-(5-chloro-2-pyridinyl)-7(S)-(4-methylpiperazin-1-yl-carbonyloxy)-6,7-dihydro-5H-pyrrolo[3,4-b]pyrazine-5-one. FIG. II schematically describes the Eszopiclone structure.

U.S. Pat. No. 6,444,673 and U.S. Pat. No. 6,864,257 describe eszopiclone as a free base form and salt-form.

U.S. Pat. No. 6,444,673 and US 2005/0043311A1 disclose preparation of eszopiclone from racemic zopiclone using D-(+)-O,O′-dibenzoyl tartaric acid. This process is followed by two crystallizations of the salt and additional crystallization of the neutralized eszopiclone free base. However, the process disclosed in U.S. Pat. No. 6,444,673 achieved a low yield of only 23%. The repetition of the crystallizations steps in order to obtain the desired optical purity also makes this process relatively expensive.

One of the methods for eszopiclone production is optical resolution of racemic zopiclone. Blaschke, G. et al., Chirality (1993) 5:419-421 discloses preparation of eszopiclone free of its enantiomer using 0.5 equivalent D-(+)-Malic acid. By this procedure the diastereomeric salt is crystallized from a mixture of methanol and acetone, the salt is then neutralized and the free base is extracted in CH₂Cl₂/ethyl acetate and precipitated by concentration of the solution. The preparation of eszopiclone by optical resolution of racemic zopiclone using D-(+)-malic acid was improved in U.S. Pat. No. 6,339,086 using one equivalent D-(+)-Malic acid. The use of D-(+)-Malic acid is very expensive. U.S. Pat. No. 6,339,086 also discloses that the undesired enantiomer could be recovered by a racemization process under basic conditions, using tertiary amines like DBU. However it is pointed in the patent that zopiclone is not stable under these conditions and racemic zopiclone is obtained in low yields.

Another method for optical resolution of racemic zopiclone described in C. F. F. Gimenez et. al., Chirality (1995) 7:267-271 by semi-preparative HPLC. A capillary electrophoresis method for zopiclone enantiomer separation is described in High, J. Resolution Chromatography (2000) 23(6):413-429.

The present invention discloses a racemization process of the R-zopiclone to racemic zopiclone which is which is then recycled into the eszopiclone preparation process.

### SUMMARY OF THE INVENTION

In one embodiment, the present invention provides a method for obtaining R-zopiclone comprising reacting a material, wherein the material comprises an acid addition salt of R-zopiclone, with at least one base in an aqueous one-phase reaction mixture to obtain R-zopiclone.

In one embodiment, the present invention provides a racemization process comprising: (a) providing a material comprising R-zopiclone; (b) mixing the material with DBU and an organic solvent having a boiling point of at least about 70° C., preferably at least about 80° C., to form a mixture; (c) heating the mixture, and then optionally cooling the heated mixture, to obtain a zopiclone racemate.

In one embodiment, the present invention provides an racemization process comprising: (A) providing a material comprising a R-zopiclone acid addition salt such as R-zopiclone malate; (B) mixing the material with water to obtain a solution; (C) neutralizing the R-zopiclone acid addition salt in the solution to form R-zopiclone, with an optional step of recovering the R-zopiclone; (D) mixing the R-zopiclone with DBU and an organic solvent having a boiling point of at least about 70° C., preferably at least about 80° C., to form a mixture; (E) heating the mixture, and then optionally cooling, the heated mixture to obtain a zopiclone racemate.

In one embodiment, the present invention provides a racemization process comprising: removing the solvent from a mother liquor comprising a R-zopiclone acid addition salt such as R-zopiclone malate, an eszopiclone acid addition salt such as eszopiclone malate and an organic solvent to obtain a mixture of the R-zopiclone acid addition salt such as R-zopiclone malate and the eszopiclone acid addition salt such as eszopiclone malate; mixing the mixture with water to obtain a solution, e.g., by adding water to obtain the solution; neutralizing the R-zopiclone acid addition salt such as R-zopiclone malate and the eszopiclone acid addition salt such as eszopiclone malate to obtain a precipitate of R-zopiclone and eszopiclone; optionally filtering the precipitate; mixing the precipitate of R-zopiclone and eszopiclone with DBU and an organic inert solvent having a boiling point of at least about 70° C., preferably at least about 80° C., e.g., by adding DBU and the organic solvent having a boiling point of at least about 70° C., preferably at least about 80° C., to the precipitate; heating; and cooling to obtain a zopiclone racemate.

In one of the embodiments, the present provides a racemization process comprising: removing the organic solvent from a mother liquor comprising R-zopiclone malate, eszopiclone malate and an organic solvent to obtain a mixture of R-zopiclone malate and eszopiclone malate; adding water to obtain a solution; neutralizing R-zopiclone malate and eszopiclone malate to obtain a precipitate of R-zopiclone and eszopiclone; filtering the precipitate; adding DBU and an organic inert solvent having a boiling point of at least 80° C.; heating; and cooling.

### DETAILED DESCRIPTION OF THE INVENTION

The term “R-zopiclone” as used herein means R-zopiclone free base.

As used herein, “eszopiclone” refers to eszopiclone free base.

As used herein, the term “organic inert solvent” means organic solvents that will not degrade R-zopiclone and eszopiclone, and will not affect the activity of DBU. Similarly, the term “organic aromatic inert solvent” refers to organic aromatic solvents that will not degrade R-zopiclone and eszopiclone, and will not affect the activity of DBU.

As used herein, “optical resolution” refers to a process of separating enantiomers in order to obtain the desired enantiomer with greater purity.

As used herein, “DBU” refers to 1,8-Diazabicyclo[5.4.0]undec-7-ene.

As used herein, the term “zopiclone racemate” refers to a mixture of R-zopiclone and eszopiclone, having an R/S ratio ranging from about 65/35 to about 35/65. Preferably, the zopiclone racemate has an R/S ratio ranging from about 55/45 to about 45/55. More preferably, the zopiclone racemate has an R/S ratio ranging from about 52/48 to about 48/52. Even more preferably, the zopiclone racemate has an R/S ratio ranging from about 51/49 to about 49/51. Most preferably, the zopiclone racemate has an R/S ratio of about 1/1.

As used herein, “racemization” of a mixture of R-zopiclone and eszopiclone means a process of converting the mixture to a zopiclone racemate, wherein the starting mixture is a material comprising R-zopiclone such that the R/S ratio of the starting mixture is above about 65/35, preferably, above about 70/30, more preferably above about 80/20. The starting mixture of R-zopiclone and eszopiclone comprises more R-zopiclone than eszopiclone, and the “racemization” reduces the relative amount of R-zopiclone and increases the relative amount of eszopiclone to form the zopiclone racemate.

As used herein, the term “isomerization” refers to a process by which one molecule is transformed into another molecule which has exactly the same atoms, but wherein these atoms have different spacial arrangement, i.e., the conversion of one compound to its optical isomer.

As used herein, “room temperature” means a temperature of about 18° C. to about 30° C., preferably 20° C. to about 25° C.

The racemization process according to the present invention can achieve a yield of more than 50%, preferably of at least about 80%. The racemization process of the present invention allows the use of a higher reaction temperature and less DBU than the prior art racemization process.

In one of the embodiments, the present invention provides a neutralization method for obtaining R-zopiclone comprising reacting a material, wherein the material comprises an acid addition salt of R-zopiclone, with at least one base in a one-phase reaction mixture comprising water to obtain R-zopiclone, wherein the material optionally further comprises an acid addition salt of eszopiclone with the molar ratio of the R-zopiclone acid addition salt and the eszopiclone acid addition salt being greater than about 65:35.

If the material comprises the acid addition salt of R-zopiclone and acid addition salt of eszopiclone with the molar ratio of greater than about 65:35, the product of the neutralization method of the present invention would have a molar ratio of R-zopiclone:eszopiclone of greater than about 65:35.

The one-phase reaction mixture preferably contains no organic solvent. Alternatively, the one-phase reaction mixture contains no water immiscible organic solvent, with the optional presence of at least one water miscible organic solvent permissible.

A preferred concentration of the material in the one-phase reaction mixture is about 0.01 to about 0.2 g per ml of water. A preferred molar ratio of the at least one base to the material is about 0.9 to about 3.

The material comprises the acid addition salt of R-zopiclone, and optionally also the acid addition salt of eszopiclone with the R/S ratio of greater than about 65:35 (mole/mole), wherein the acid is organic or inorganic, and the acid is, preferably, an optically active acid.

The acid addition salt(s) can be water soluble. Most preferably, the acid addition salt(s) is a water soluble salt(s) of an optically active acid, such as a D-(+)-malate salt. Additional examples of organic optically active acid addition salts are: D-(+)-O,O-ditoluoyl-tartarate, D-(+)-tartarate, D-(+)-mandelate, and D-(+)-O,O′-dibenzoyl tartarate. If the salt is not water soluble, a slurry is obtained. If the salt is water soluble, a solution is obtained.

Preferably, the temperature during reaction of the at least one base with the material is of about 5° C. to about 60° C., more preferably, of about room temperature to about 50° C., more preferably, about room temperature to about 40° C., and, most preferably, about room temperature.

Preferably, prior to the base addition, activated carbon is added to the solution. Preferably, the solution containing the activated carbon is stirred. Preferably, the stirring is for about ½ hour to about 5 hours, more preferably, for about ½ hour to about 3 hours, and most preferably, for about 1 hour. Preferably, after the stirring and prior to the base addition, the activated carbon is removed. Preferably, the removal is by filtration.

Preferably, the at least one base is a mild base, organic or inorganic. More preferably, the mild base is an inorganic base. The at least one base can be selected from the group consisting of ammonia, alkaline earth metal hydroxide, alkali metal hydroxide, alkali metal carbonates, alkaline earth metal carbonates, alkali metal bicarbonates, alkaline earth metal bicarbonates and amines. Preferably, the alkali metal is selected from the group consisting of potassium and sodium. Preferably, the carbonate is selected from the group consisting of potassium carbonate and sodium carbonate. Preferably, the bicarbonate is sodium bicarbonate or potassium bicarbonate. The at least one inorganic base may be added as a solid or an aqueous solution. Preferably, the at least one inorganic base is added as an aqueous solution. Preferably, the aqueous solution of the at least one inorganic base is added gradually. Preferably, the aqueous solution of the at least one inorganic base is added during a period of about 1 hour to about 3 hours, more preferably, about 2 hours to about 3 hours.

An ideal pH after addition of the base is about 7 to about 12, preferably about 8.

After the base addition a suspension is obtained. Preferably, the suspension is stirred for a period of time after addition of the base. Preferably, the stirring is for about 1 hour to about 24 hours, and more preferably, for about 2 hours to about 4 hours.

Preferably, the obtained R-zopiclone is further isolated. Preferably, the isolation is by filtration. The isolated R-zopiclone may be further washed and dried. Preferably, the washing is with water. Preferably, the drying is at a temperature of about 30° C. to about 80° C., more preferably, about 40° C. to about 70° C., most preferably, about 50° C., under vacuum (less than 100 mm Hg). Preferably, the drying is for about 6 hours to about 16 hours.

In a preferred embodiment, the present invention provides a process for preparing a mixture of R-zopiclone and eszopiclone wherein the molar ratio of R-zopiclone and eszopiclone or the weight ratio of R-zopiclone and eszopiclone is greater than about 65/35. The process comprises: neutralizing a mother liquor comprising R- and S-zopiclone acid addition salts in a molar ratio of R-zopiclone and eszopiclone or a weight ratio of R-zopiclone and eszopiclone of greater than about 65/35, in water and a base, preferably in a one-phase system such as in the absence of any water immiscible organic solvent, e.g., in the absence of any organic solvent. The mother liquor is typically obtained from an optical resolution process of racemic zopiclone, wherein the obtained eszopiclone is removed, for example, by filtration, and the remaining substance is used as the mother liquor in the neutralization step of the process of the present invention, wherein the mother liquor contains R- and S-zopiclone acid addition salts in a ratio of R-zopiclone and eszopiclone above about 65/35 (w/w, or mole/mole). Preferably, the optical resolution process comprises the use of an acid addition salt, wherein the acid is a chiral acid. Preferably, the chiral acid is D-(+)-malic acid.

Preferably, the acid addition salt is a D-(+)-malate salt. Preferably, the neutralization is done at room temperature.

The present invention provides a racemization process comprising: (a) providing a material comprising R-zopiclone; (b) mixing the material with DBU and at least one organic solvent having a boiling point of at least about 70° C. to form a mixture, and (c) heating, with optional cooling after heating, to obtain a zopiclone racemate.

Optionally, the starting material comprising R-zopiclone used in step (a) further comprises eszopiclone, wherein the amounts of R-zopiclone and eszopiclone in the starting material are not the same and wherein the R/S ratio is greater than about 65/35 (w/w, or mole/mole).

Preferably, the starting material comprising R-zopiclone is obtained from a mother liquor comprising R- and S-zopiclone acid addition salts, and optionally an organic solvent or water, obtained from optical resolution of racemic zopiclone, optionally using a chiral salt. If the mother liquor contains an organic solvent, the organic solvent is removed from the mother liquor to obtain the mixture of R-zopiclone acid addition salt and eszopiclone acid addition salt, wherein the mixture is dissolved in any water already present in the mother liquor or in water optionally added to obtain an aqueous solution of the R-zopiclone acid addition salt and eszopiclone acid addition salt. If the mother liquor contains water and no organic solvent, an aqueous solution of the R-zopiclone acid addition salt and eszopiclone acid addition salt is formed. The R- and S-zopiclone acid addition salts in the solution are then neutralized to obtain a precipitate of a mixture of R-zopiclone and eszopiclone. The precipitate is filtered. The filtered precipitate is then used as the starting material in the process of the present invention. Preferably, the R- and S-zopiclone acid addition salts are R- and S-zopiclone malate. The organic solvent may be acetone, methanol and mixtures thereof. Preferably, the neutralization is by adding sodium carbonate, potassium carbonate, sodium bicarbonate or potassium bicarbonate. Preferably, the amount of the base is of about 0.9 to about 1.2 equivalents with reference to the acid addition salt being neutralized.

Preferably, the at least one organic solvent having a boiling point of at least about 70° C. has a boiling point of at least about 80° C. The at least one organic solvent having a boiling point of at least about 70° C. used in the reaction involving DBU is preferably at least one organic inert solvent. The at least one organic inert solvent having a boiling point of at least 70° C. can be at least one organic aromatic inert solvent, cyclohexane, methyl ethyl ketone or acetonitrile. Preferably, the at least one organic inert solvent having a boiling point of at least 70° C. is selected from the group consisting of: toluene, xylene, chlorobenzene, m-dichlorobenzene, o-dichlorobenzene, p-dichlorobenzene, bromobenzene, cyclohexane, methyl ethyl ketone and acetonitrile.

The amount of the at least one organic solvent having a boiling point of at least about 70° C. used can be, for example, about 2 to 50 ml per gram, preferably about 5 to 20 ml per gram, and most preferably about 10 ml per gram, of the starting material comprising R-zopiclone, or of the starting material comprising R-zopiclone and eszopiclone.

Typically, the heating is to a temperature of about 50° C. to about reflux, preferably to a temperature of about 50° C. to about 110° C., depending on the kind of the at least one organic solvent having a boiling point of at least about 70° C. used. More preferably, the heating is to a temperature of about 80° C. to about 90° C. When toluene is used as the at least one organic inert solvent the heating temperature may vary between 80° C. to about reflux, usually for about 5 hours to about 14 hours, more preferably, for about 14 hours. When xylene is used as the at least one organic inert solvent, the heating is preferably to a temperature of about 70° C. to about 1000° C., for about 1 to about 2 hours. More preferably, when xylene is the at least one organic inert solvent the heating starts from a temperature of about 70° C., for about 30 minutes, and then goes up to a temperature of about 100° C. for about 1 hour to about 5 hours.

When acetonitrile is used as the at least one organic inert solvent, the heating is preferably to a temperature of about 50° C. to about 80° C., for about 1 hour to about 24 hours.

Preferably, the DBU is used in a concentration of about 0.5% to about 10%, preferably, of about 1% to about 5% w/w, and more preferably, of about 1% to about 2% w/w, relative to the starting material comprising R-zopiclone, or relative to the starting material comprising R-zopiclone and eszopiclone.

The cooling step is done to about 50° C. to about 20° C., more preferably, to about room temperature. When the reaction is done at about 50° C. the cooling is to about 20° C.

Preferably, the obtained zopiclone racemate is recovered or isolated by a procedure such as filtration, centrifugation and/or recrystallization known in the art. Preferably, the recovery comprises: filtering, washing and drying. Preferably, the washing is with the same organic inert solvent used in the process. Preferably, the drying is performed at a temperature of about 40° C. under vacuum.

Preferably, the obtained zopiclone racemate is in a ratio of about 60:40, more preferably about 50:50, of R-zopiclone:eszopiclone, wherein the amounts of R-zopiclone and eszopiclone used to calculate the ratio are expressed in the same unit.

The present invention also provides a process of making eszopiclone, comprising converting the zopiclone racemate obtained by any one of the processes of the present invention described above with an optical resolution procedure known in the art. For instance, the zopiclone racemate can be converted to eszopiclone by using about one equivalent of D-(+)-malic acid

The disclosures of the prior art references mentioned in the application are herein incorporated by reference in their entirety.

With the present invention described above, some of the embodiments of the present invention are exemplified below. The examples are for demonstration purposes and do not represent the full scope of the invention. It will be apparent to one skilled in the art that the examples presented below can be modified without departing from the purpose and interest of the present invention.

### EXAMPLES

### Example 1

### Neutralization of the Mixture R-Zopiclone Malate and Eszopiclone Malate

The mixture of R-zopiclone and eszopiclone free base was prepared by neutralization of a mother liquor after separation of eszopiclone malate, wherein the solvents (methanol and acetone) in the mother liquor were evaporated under vacuum, the residue was dissolved in water, neutralized with potassium carbonate, filtered and dried.

### Example 2

### Racemization in Toluene at 80° C.

A solution of R-zopiclone and eszopiclone (5.0 g R/S ratio 9/1 by HPLC analysis) and DBU (400 mg) in 100 ml of toluene was heated at 80° C. for 14 hours. After slow cooling to room temperature the obtained solid was filtered, washed with toluene, and dried under vacuum at 40° C. overnight to give 2.6 g (52%) of racemic Zopiclone (R/S ratio 51.62/48.18, chemical purity 99.39% w/w by HPLC analysis).

### Example 3

### Racemization in Boiling Toluene

A solution of R-zopiclone and eszopiclone (5.0 g R/S ratio 9/1 by HPLC analysis) and DBU (100 mg) in 50 ml of toluene was heated at reflux for 14 hours. After slow cooling to room temperature the obtained solid was filtered, washed with toluene, dried under vacuum at 40° C. overnight to give 3.7 g (72%) of racemic Zopiclone (R/S ratio 50.24/49.76, chemical purity 98.98% w/w by HPLC analysis).

### Example 4

### Racemization in Xylene

To the solution of R-zopiclone and eszopiclone (3.02 g), obtained by neutralization in water of the material from the mother liquids of the optical resolution of zopiclone, followed by filtration, in xylene (30 ml) was added DBU (0.12 g, 4% w/w); the solution was heated at 70° C. for about 30 min. and then at 100° C. for 1 hour. The solution was cooled to the room temperature and the obtained solid was filtrated, washed with xylene and dried to affords the product racemic zopiclone (2.48 g, yield 82%, R/S ratio 1:1).

### Example 5

### Racemization in Acetonitrile

In a three necked flask were charged, under nitrogen flow, acetonitrile (10 ml), R-zopiclone/eszopiclone mixture (1 g) obtained from the mother liquid of the optical resolution by neutralization in water and filtration, and 10% w/vol. solution of DBU in acetonitrile (0.1 ml). The obtained solution was heated at about 80° C. and stirred at this temperature for 1.25 hour. The whole was cooled to room temperature and then the reaction vessel was kept in a refrigerator. The solid was filtrated, washed with cold acetonitrile and dried in vacuum oven to afford the product (0.66 g, yield 66%, R/S ratio 50.6:49.4, chemical purity 97.9% w/w by HPLC analysis).

We claim:

1. A neutralization method for obtaining R-zopiclone comprising reacting a material, wherein the material comprises an acid addition salt of R-zopiclone, with at least one base in a one-phase reaction mixture comprising water to obtain R-zopiclone.

2. The method of claim 1, wherein the concentration of the material in the one-phase reaction mixture is about 0.01 to about 0.2 g per ml of water.

3. The method of claim 1, wherein the material further comprises an acid addition salt of eszopiclone with the molar ratio of the acid addition salt of R-zopiclone and the acid addition salt of eszopiclone being greater than about 65:35, and wherein the acid in the acid addition salt of R-zopiclone and the acid in the acid addition salt of eszopiclone are the same or different.

4. The method of claim 3, wherein the acid in the acid addition salt of R-zopiclone and the acid in the acid addition salt of eszopiclone are the same.

5. The method of claim 3, wherein the acid in the acid addition salt of R-zopiclone and/or the acid in the acid addition salt of eszopiclone is an optically active acid.

6. The method of claim 5, wherein the optically active acid is selected from the group consisting of D-(+)-malic acid, D-(+)-O,O-ditoluoyl-tartaric acid, D-(+)-tartaric acid, D-(+)-O,O′-dibenzoyl tartaric acid and D-(+)-mandelic acid.

7. The method of claim 6, wherein the optically active acid is D-(+)-malic acid.

8. The method of claim 1, wherein the process is carried out in the absence of any water immiscible organic solvent.

9. The process of claim 8, wherein the process is carried out in the absence of any organic solvent.

10. The method of claim 1, wherein the at least one base is selected from the group consisting of ammonia, alkali metal hydroxides, alkaline earth metal hydroxides, alkali metal carbonates, alkaline earth metal carbonates, alkali metal bicarbonates, alkaline earth metal bicarbonates and amines.

11. The method of claim 10, wherein the at least one base is selected from the group consisting of alkali metal hydroxides, alkali metal carbonates and alkali metal bicarbonates.

12. The method of claim 11, wherein the at least one base is selected from the group consisting of potassium carbonate, sodium carbonate, potassium bicarbonate and sodium bicarbonate.

13. The method of claim 1, wherein the pH of the one-phase reaction mixture after the addition of the at least one base is about 7 to about 12.

14. The method of claim 13, wherein the pH is about 8.

15. The method of claim 1, wherein the material is reacted with the at least one base at a temperature of about 5° C. to about 60° C.

16. The method of claim 15, wherein the temperature is about room temperature to about 50° C.

17. The method of claim 16, wherein the temperature is about room temperature.

18. The method of claim 1, wherein prior to reacting the material with the at least one base, activated carbon is added to the mixture.

19. The method of claim 1, wherein the obtained R-zopiclone is recovered.

20. The method of claim 19, wherein the recovered R-zopiclone is washed and dried.

21. The method of claim 20, wherein the recovered R-zopiclone is washed with water.

22. The method of claim 3, wherein the molar ratio of the R-zopiclone and eszopiclone obtained is greater than about 65:35 (R-zopiclone:eszopiclone).

23. The method of claim 3, wherein the material is a mother liquor obtained by a process comprising:
conducting optical resolution of racemic zopiclone to obtain a product comprising eszopiclone; and
removing some of the eszopiclone to obtain a substance comprising R- and S-zopiclone addition salts in a molar ratio of greater than about 65:35, wherein the substance is provided as the mother liquor.

24. The method of claim 23, wherein the optical resolution of the racemic zopiclone is conducted using a chiral acid.

25. The method of claim 24, wherein the chiral acid is D-(+)-malic acid.

26. A racemization process comprising:
a. providing a material comprising R-zopiclone;
b. mixing the material with DBU and at least one organic solvent having a boiling point of at least about 70° C. to form a mixture; and
c. heating the mixture to obtain a zopiclone racemate.

27. The process of claim 26, further comprising cooling the heated mixture in step (c) to obtain the zopiclone racemate.

28. The process of claim 26, wherein the material in step (a) further comprises eszopiclone at a molar ratio of R-zopiclone:eszopiclone of greater than about 65:35.

29. The process of claim 28, further comprising the following steps before step (a): (A) providing a material comprising a R-zopiclone acid addition salt and an eszopiclone acid addition salt at a molar ratio greater than about 65:35 (R-zopiclone acid addition salt:eszopiclone acid addition salt); and (B) neutralizing the R-zopiclone acid addition salt and eszopiclone acid addition salt to form R-zopiclone and eszopiclone, wherein the R-zopiclone and eszopiclone is provided in the material in step (a).

30. The process of claim 29, wherein the neutralizing in step (B) is conducted with at least one base and water in a one-phase reaction mixture.

31. The process of claim 30, wherein the one-phase reaction mixture comprises no water immiscible organic solvent.

32. The process of claim 30, wherein the at least one base is selected from the group consisting of sodium carbonate, potassium carbonate, sodium bicarbonate and potassium bicarbonate.

33. The process of claim 29, wherein the material in step (A) comprises a mother liquor obtained by a process comprising:
conducting optical resolution of racemic zopiclone to obtain a product comprising eszopiclone; and
removing some of the eszopiclone to obtain a substance comprising R- and S-zopiclone acid addition salts in a molar ratio of greater than about 65:35, wherein the substance is provided as the mother liquor.

34. The process of claim 33, wherein the optical resolution of the racemic zopiclone is conducted using a chiral acid.

35. The process of claim 34, wherein the chiral acid is D-(+)-malic acid.

36. The process of claim 29, wherein the R-zopiclone acid addition salt is R-zopiclone malate and the eszopiclone acid addition salt is eszopiclone malate.

37. The process of claim 33, wherein the material further comprises at least one organic solvent in step (A), and wherein the at least one organic solvent is removed before the neutralization in step (B).

38. The process of claim 37, wherein the at least one organic solvent is methanol, acetone or a mixture thereof.

39. The process of claim 29, wherein the material in step (A) is mixed with water to form a solution before step (B).

40. The process of claim 29, wherein the R-zopiclone and eszopiclone formed in step (B) are recovered by filtration.

41. The process of claim 29, wherein the R-zopiclone acid addition salt and eszopiclone acid addition salt in the material is at a molar ratio of about 80:20 or higher.

42. The process of claim 26, wherein the at least one organic solvent having a boiling point of at least about 70° C. is inert.

43. The process of claim 26, wherein the at least one organic solvent has a boiling point of at least about 80° C.

44. The process of claim 26, wherein the at least one organic solvent having a boiling point of at least about 70° C. is selected from the group consisting of organic aromatic inert solvents, cyclohexane, methyl ethyl ketone and acetonitrile.

45. The process of claim 44, wherein the at least one organic solvent having a boiling point of at least about 70° C. is selected from the group consisting of toluene, xylene, chlorobenzene, m-dichlorobenzene, o-dichlorobenzene, p-dichlorobenzene, bromobenzene, cyclohexane, methyl ethyl ketone and acetonitrile.

46. The process of claim 26, wherein the heating is to a temperature ranging from about 50° C. to about reflux.

47. The process of claim 45, wherein the at least one organic solvent is toluene.

48. The process of claim 47, wherein the heating is at a temperature ranging from about 80° C. to about reflux.

49. The process of claim 45, wherein the at least one organic solvent is xylene.

50. The process of claim 49, wherein the heating is at a temperature ranging from about 70° C. to about 100° C.

51. The process of claim 45, wherein the at least one organic solvent is acetonitrile.

52. The process of claim 51, wherein the heating is at a temperature ranging from about 50° C. to about 80° C.

53. The process of claim 26, wherein the volume of the at least one organic solvent having a boiling point of at least about 70° C. used is about 2 to 50 ml per gram of the R-zopiclone in the material in step (a).

54. The process of claim 53, wherein the volume of the at least one organic solvent having a boiling point of at least about 70° C. used is about 5 to 20 ml per gram of the R-zopiclone in the material in step (a).

55. The process of claim 54, wherein the volume of the at least one organic solvent having a boiling point of at least about 70° C. used is about 10 ml per gram of the R-zopiclone in the material in step (a).

56. The process of claim 28, wherein the volume of the at least one organic solvent having a boiling point of at least about 70° C. used is about 2 to 50 ml per gram of the R-zopiclone and eszopiclone in the material in step (a).

57. The process of claim 56, wherein the volume of the at least one organic solvent having a boiling point of at least about 70° C. used is about 5 to 20 ml per gram of the R-zopiclone and eszopiclone in the material in step (a).

58. The process of claim 57, wherein the volume of the at least one organic solvent having a boiling point of at least about 70° C. used is about 10 ml per gram of the R-zopiclone and eszopiclone in the material in step (a).

59. The process of claim 27, wherein the heated mixture in step (c) is cooled to a temperature ranging from about 20° C. to about 50° C. to obtain the zopiclone racemate.

60. The process of claim 26, wherein the DBU is used in a concentration of about 1% to about 10% w/w relative to the R-zopiclone in the material.

61. The process of claim 28, wherein the DBU is used in a concentration of about 1% to about 10% w/w relative to the R-zopiclone and eszopiclone in the material.

62. The process of claim 26, wherein the obtained zopiclone racemate has an R/S ratio ranging from about 55/45 to about 45/55.

63. The process of claim 62, wherein the obtained zopiclone racemate has an R/S ratio ranging from about 52/48 to about 48/52.

64. The process of claim 63, wherein the obtained zopiclone racemate has an R/S ratio about 1/1.

65. The process of claim 26, wherein the yield of the zopiclone racemate is at least about 50%.

66. The process of claim 65, wherein the yield of the zopiclone racemate is at least about 80%.

67. A process for preparing eszopiclone, comprising
obtaining the zopiclone racemate as prepared by the process according to claim 26; and
converting the zopiclone racemate to eszopiclone by optical resolution.

68. The process of claim 67, wherein the optical resolution is performed by using about one equivalent of D-(+)-malic acid.
